(19) United States
(12) Patent Application Publication
(10) Pub. No.: US 2024/0358872 A1
(43) Pub. Date: Oct. 31, 2024

(54) METHOD FOR IDENTIFYING AND REPROCESSING A MEDICAL DEVICE, AND REPROCESSING MACHINE AS SUCH

(71) Applicant: Ecolab USA Inc., Saint Paul, MN (US)

(72) Inventors: Sébastien RUIZ, Saint-Victoret (FR); Eric PENDARIES, La Ciotat (FR); Laurent BERENGUER, Aubagne (FR)

(21) Appl. No.: 18/646,155

(22) Filed: Apr. 25, 2024

Related U.S. Application Data
(60) Provisional application No. 63/499,086, filed on Apr. 28, 2023.

(51) Int. Cl. A61L 2/22; A61B 90/98

(52) U.S. Cl. CPC A61L 2/22; A61B 90/98; A61L 2202/121; A61L 2202/122; A61L 2202/24

(57) ABSTRACT

Systems and techniques for reprocessing a medical device equipped with an electromagnetic tag can include opening an access door of a reprocessing machine and passing the medical device through an entry-opening towards an inside of a washing chamber of the reprocessing machine. The systems and techniques can also involve energizing an electromagnetic tag carried by the medical device with an energizing power source of the reprocessing machine. As the medical device is passing through the entry-opening (EO) towards the inside of the washing chamber and in response to the energizing, the system can receive an identification electromagnetic signal transferred by the energized electromagnetic tag with a receiver the reprocessing machine. Information associated with the identification electromagnetic signal can be used to select and/or control washing parameters of the reprocessing machine.

### RELATED APPLICATION

This application claims priority to U.S. Provisional Application No. 63/499,086, filed on Apr. 28, 2023, the entire contents of which are incorporated herein by reference.

### TECHNICAL FIELD

This disclosure pertains to the field of machines for reprocessing medical devices, such as endoscope. Such reprocessing machine comprises a tank delimiting a washing chamber, and an access-door configured to close an entry-opening of the tank for the medical device, and a fluidic circuit comprising one or several pumps, one or several valves. Such reprocessing machine are typically called automatic endoscope reprocessing (“AER”) in the field.

Such a machine, once the medical apparatus is inserted in the tank, enables to clean the outer surfaces by spraying a cleaning solution, or by immersing the medical device the cleaning solution. The reprocessing machine can typically be configured to clean the inner surfaces of the medical device. For this purpose the inner lumens of the medical device can be coupled by flexibles pipe to coupling holes of the reprocessing machine, enabling a circulation of the cleaning solution through the inner limen.

For responding to security reasons, it is known in the prior art to equip:
the medical device with an electromagnetic tag containing information on the medical device,
the reprocessing machine with a reader comprising a power energizing source allowing to energize the electromagnetic tag and, in response to energization, a receiver enabling to receive an identification signal transferred by the tag.

### BACKGROUND ART

According to inventor knowledge, and according to a first prior art, the reprocessing machine is equipped with a proximity reader technology (RFID) and have the reader of the tag located, behind a side wall of the tank.

Once the medical device is inserted into the washing chamber, in order to allow the reader to read the tag, such a first prior art forces the operator to grab the medical device from a tray, and to displace it against the side wall where the reading zone of the reader lies.

According to inventor, such a first prior art has the following drawbacks:
it is mandatory to have a side wall in a material such as plastic, permeable to the electromagnetic signal of the reader and the tag: in other word having a metallic side wall is forbidden, as the electromagnetic signal of the reader and the tag cannot go through it, and thus read the tag, when the metallic wall separate the reader and the tag,
it is mandatory for the operator to grab manually the medical device, which is not ideal since it is typically covered with infectious organic material.

According to EP3226918 A1, it is known a second prior art where a preparation system comprises a preparation device, the preparation chamber of which is closed by a door.

A sensor which operates in a contactless manner is designed to detect a machine-readable ID feature attached to medical device to carry out a movement analysis on the basis of time-dependent location coordinates. It is determined whether the medical device is moving towards the door of the preparation device, and if so, a door opening signal is generated and the treatment chamber is made accessible by opening the door.

ID feature may be a RFID tag, and in particularly a long ranger RFID tag so as to enable the movement analysis when the ID feature is displaced towards the door.

According to inventor, such a second prior art using long range RFID technology is not ideal for security reason as it enables a user to insert a second medical device into the treatment chamber of the preparation device, once the door is opened, and whereas the door has opened due to detection of the ID feature of a first medical device. It thus can lead to serious error in the traceability.

This security problem is particularly exacerbated in limited environment, typically in small room.

### SUMMARY

According to a first aspect it is proposed a method for identifying and reprocessing a medical device, the medical device being equipped with an electromagnetic tag, the electromagnetic tag comprising an antenna and a memory comprising information on the medical device, said method comprising:
/A/ opening an access door of a reprocessing machine comprising a tank delimiting a washing chamber, the access door being configured to close an entry-opening of the tank for passing the medical device through the entry-opening;
/B/ as the medical device is passing through the entry-opening and towards an inside of the washing chamber, energizing the electromagnetic tag with an energizing power source of the reprocessing machine located at the access-door, and outside of the washing chamber;
/C/ as the medical device is passing through the entry-opening and towards the inside of the washing chamber and in response to the energizing, receiving an identification electromagnetic signal transferred by the energized electromagnetic tag with a receiver of the reprocessing machine located at the access-door and outside of the washing chamber.

The following features of the first aspect, can be optionally implemented, separately or in combination one with the others:

The method further comprises supporting the medical device with a carrier device and sliding the carrier device along supporting tracks inside the tank for passing the medical device through the entry opening.

The medical device can be an endoscope having a plurality of lumens,
and wherein the method further comprises, before the opening the access door;
coupling the plurality of lumens on a coupling block of the carrier device, the coupling block having a plurality of through-holes that have an inlet side that are fluidly coupled to corresponding ones of the plurality of lumens and an outlet side configured to couple to a plurality of holes of the reprocessing machine.

The method can further comprise;
amplifying a signal of the energizing power source with a relay antenna of the carrier device, the relay antenna being located in between the identification electromagnetic tag and the energizing power source, as the medical device is passing through the entry-opening and towards the inside of the washing chamber, so as to generate an amplified energizing signal reaching the electromagnetic tag.

The electromagnetic tag is a RFID Tag, in particularly RFID Tag can be a short-range RFID Tag functioning at 13.56 MHZ.

The method can further comprise:
/D/ closing the access door in response passing the medical device through the entry opening and towards the inside of the washing chamber;
/E/ in response to closing the door, washing the medical device by applying a washing cycle by controlling a fluidic circuit comprising one or several pumps, one or several valves and a sprayer device located inside said washing chamber.

The method can further comprise:
/F/ extracting the washed medical device through an exit door located opposite to the access door relative to the washing chamber.

The method can further comprise, in response to receiving an identification electromagnetic signal, reading the identification information of the medical device, and comparing the identification information to stored information to determine if the medical device is authorized to be cleaned.

The method can further comprise, in response to receiving an identification electromagnetic signal, reading the identification information of the medical device, and comparing the identification information to stored information to select wash parameters associated with the specific medical device inserted into the washing chamber.

The method can further comprise:
displaying a selected wash cycle associated with the selected wash parameters on a screen of a user interface of the reprocessing machine and waiting for a user confirmation to initiate the selected wash cycle;
initiating the selected wash cycle, in response to a user confirmation on the user interface.

The method can further comprise:
in response to a user discarding confirmation of the selected wash cycle, displaying on the screen of the user interface changes in wash parameters of the wash cycle, and allowing the user to select one or several changes in the parameters of the wash cycle,
initiating the wash cycle, in response to change in the parameter, after a user confirmation on the user interface.

According to a second aspect it is proposed a reprocessing machine configured to clean a medical device, the medical device being equipped with an electromagnetic tag, the reprocessing machine comprising:
a tank delimiting a washing chamber, and an access door configured to close an entry-opening of the tank for the medical device,
a fluidic circuit comprising one or several pumps, one or several valves and a sprayer device inside the washing chamber,
an energizing power source configured to energize the identification electromagnetic tag, and a receiver configured to receive an identification electromagnetic signal of the electromagnetic tag, in response to the energization, the energizing power source and the receiver being located at the access-door, outside the washing chamber,
a control device including a processor, a storage and an instruction set configured to implement the method according to the present disclosure, including receiving an identification electromagnetic signal, as the access-door is opened, during displacement of the medical device through said entry-opening towards the inside the washing chamber.

According to a third aspect it is proposed a reprocessing machine configured to clean a medical device, the medical device being equipped with an electromagnetic tag, the reprocessing machine comprising:
a tank delimiting a washing chamber, and an access door configured to close an entry-opening of the tank for the medical apparatus,
a fluidic circuit comprising one or several pumps, on a several valve and a sprayer device inside the washing chamber,
an energizing power source configured to energize the identification electromagnetic tag, and a receiver configured to receive an identification electromagnetic signal of the electromagnetic tag, in response to the energization, the energizing power source and the receiver being located at the access-door outside the washing chamber,
a control device including a processor and a storage and an instruction set configured to cooperate with the processor and the storage to:
/a/ open the access door of a reprocessing machine;
/b/ energize the identification electromagnetic tag with the energizing power source, as the medical device passes through the entry-opening and towards the inside of the washing chamber and,
/c/ receive an identification electromagnetic signal transferred by the energized electromagnetic tag with the receiver, as the medical device passes through the entry-opening and towards the inside of the washing chamber, and in response to the energization of the identification electromagnetic tag.

The following features of the second or third aspect, can be optionally implemented, separately or in combination one with the others:

The reprocessing can further comprise a carrier device for the medical device and supporting tracks inside the tank for passing the medical device through the entry-opening towards the inside of the washing chamber.

The reprocessing can further comprise a relay antenna of the carrier device, the relay antenna being located in between the identification electromagnetic tag and the energizing power source, as the medical device is passing through the entry-opening and towards the inside of the washing chamber, so as to generate an amplified energizing signal reaching the electromagnetic tag.

The medical device can be an endoscope having a plurality of lumens, and wherein the carrier device can have a coupling block, the coupling block having a plurality of through-holes that have an inlet side that are fluidly coupled to corresponding ones of the plurality of lumens and an outlet side configured to couple to a plurality of holes of the reprocessing machine.

The sprayer device can comprise a rotating sprayer arm, the rotating sprayer arm being located:
below the carrier device supported by the supporting tracks and,
above a level of the energizing power source and the receiver.

The energizing power source can be located at a vertical distance (L) from a supporting surface of the carrier device supported by the tracks, the vertical distance being greater than 4 cm and below 10 cm.

The energizing power source and the receiver can be fixed to a lower beam of a door frame of the access-door.

According to a fourth aspect it is proposed a computer program comprising instructions which, when the program is executed by a control unit of a reprocessing machine comprising a processor and a storage, cause the computer to carry out the method according to the present disclosure.

According to a sixth aspect it is proposed a computer-readable medium having stored thereon the computer program of the present disclosure.

According to a seventh aspect it is proposed a non-transitory machine-readable storage medium encoded with instructions executable by a processor, the machine-readable storage medium comprising:
instructions to open the access door of a reprocessing machine,
instructions to energize the electromagnetic tag with the energizing power source, as the medical device passes through the entry-opening and towards the inside of the washing chamber,
instructions to receive an identification electromagnetic signal transferred by the energized electromagnetic tag with the receiver, as the medical device s passes through the entry-opening and inside the washing chamber, and in response to the energization of the identification electromagnetic tag.

The following features of the seventh aspect, can be optionally implemented, separately or in combination one with the others:

The machine-readable storage medium further can further comprise instructions to;
close the access door,
wash the medical device by applying a washing cycle by controlling a fluidic circuit comprising one or several pumps, on or several valves and a sprayer device located inside said washing chamber.

The machine-readable storage medium further can further comprise instructions to;
read the identification information of the medical device, in response to receiving an identification electromagnetic signal, and,
compare the identification information to stored information to determine if the medical device is authorized to be cleaned.

The machine-readable storage medium further can further comprise instructions to;
read the identification information of the medical device, in response to receiving an identification electromagnetic signal and,
compare the identification information to stored information to select wash parameters associated with the specific medical device inserted into the washing chamber.

The machine-readable storage medium further can further comprise instructions to;
display a selected wash cycle associated with the selected wash parameters on a screen of a user interface of the reprocessing machine, and waiting for a user confirmation to initiate the selected wash cycle;
initiate the selected wash cycle, in response to a user confirmation on the user interface.

The machine-readable storage medium further can further comprise instructions to update a traceability numerical file by changing a status of a corresponding identified medical device to a cleaned medical device.

### BRIEF DESCRIPTION OF DRAWINGS

Other features, details and advantages will be shown in the following detailed description and on the figures, on which:

FIG. 1 is a schematical view of the reprocessing machine, during insertion of the medical device through an entry opening, which is opened by an access door, the medical device being supported by a carrier device, in a position where a reader (comprising a energizing power source and a receiver) is configured to read a tag of the medical device, the reader located outside the washing chamber, at the level of the access door to enable reading the tag, and especially without having the electromagnetic signal between the tag and the reader having to go through a wall of the tank.

FIG. 2 is perspective view of a non-limiting example of the present disclosure, once the access-door is opened, the operator initiating an insertion of a medical device supported by a carrier device, by manually grabbing the carrier device.

FIG. 3 is a consecutive view of FIG. 2, as the carrier device is further slide towards the tag reader inside of the tank, in a position where the electromagnetic tag of the medical device, and the reader are aligned, in particularly vertically, allowing a reading of the information contained in the electromagnetic tag.

FIG. 4 is a consecutive view of FIG. 3, as the medical device and the carrier device are inserted in the washing chamber, the carrier device being supporting by supporting tracks, the view showing and upper spraying device, and a lower spraying device which is a rotative sprayer arm located below the carrier device, and above the lover of the reader, a vertical distance L separating the reader and the supporting surface of the carrier device.

FIG. 5 is a perspective showing a coupling block on a side of the carrier device that comprises a plurality of through-holes that have an inlet side that are indented to be fluidly coupled to corresponding ones of the plurality of lumens of the medical device and an outlet side configured to couple to a plurality of holes of a corresponding block the reprocessing machine, and in a position where the coupling block of the carrier device and the corresponding block of the reprocessing machine are aligned to enable coupling of the outlet side.

FIG. 6 is a diagram showing blocks of the method according to the present disclosure.

FIG. 7 is a diagram showing blocks of the method according to an example.

### DETAILED DESCRIPTION

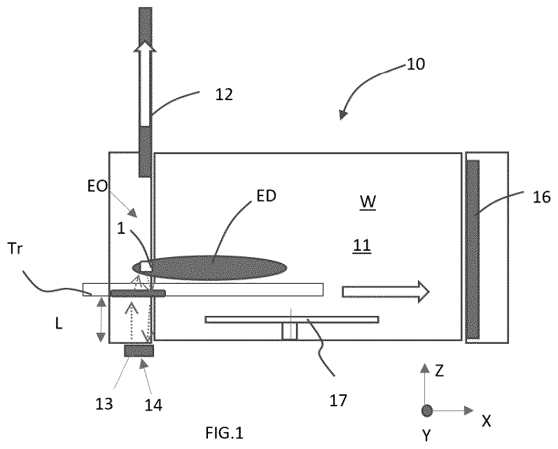

As shown on FIG. 1, the present disclosure relates to a reprocessing machine 10 configured to clean a medical device. The medical device can be an endoscope.

The medical device is equipped with an electromagnetic tag.

The electromagnetic tag comprises an antenna and a memory comprising information on the medical device. The information may comprise specific data enabling to identify the medical apparatus. The information may also contain specific data enabling to identify one or several patients on which the medical device has be used by a doctor. The information contained in the tag can be used to secure a traceability procedure.

The reprocessing machine comprises:
a tank 11 delimiting a washing chamber, and an access door 12 configured to close an entry-opening of the tank for the medical device ED,
a fluidic circuit comprising one or several pumps, one or several valves and a sprayer device inside the washing chamber.

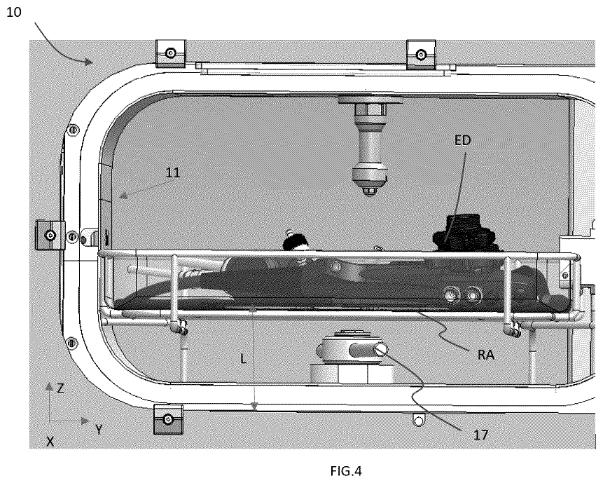

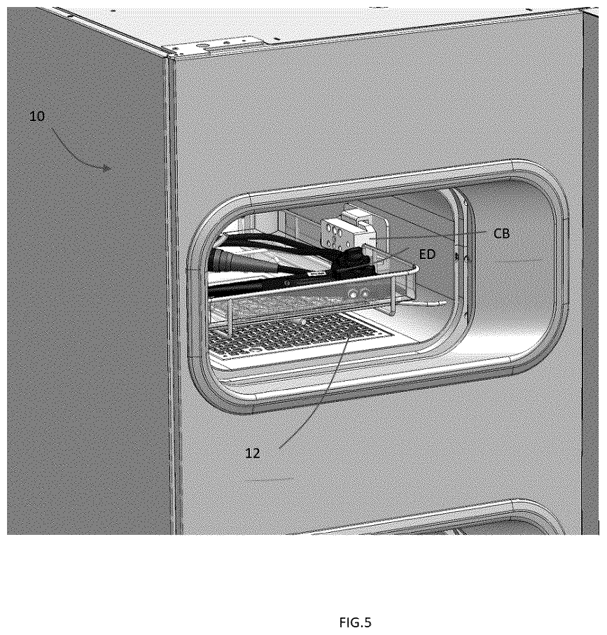

The access door is configured to pass from an open position to a close position, and to the close position to the open position. The access-door can for example be a pivoting or a sliding door, or even a more having a more complex cinematic of opening and closing. The sliding door can slide in a vertical direction Z, for instance according to FIG. 1, or in a horizontal direction, for instance according to direction Y on FIG. 4.

The tank 11 can be in plastic, or in metal, or other material. According to an advantageous embodiment the tank is in metal. A metallic tank may be advantageous compared to a plastic tank. For instance, it may enable to reach higher temperature compared to temperature reachable with a plastic tank.

The reprocessing machine further comprises an energizing power source 13 configured to energize the identification electromagnetic tag 1, and a receiver 14 configured to receive an identification electromagnetic signal of the electromagnetic tag, in response to the energization.

The energizing power source 13 and the receiver 14 are located, typically locally on a same position, at the access-door 12, outside the washing chamber W. The energizing power source 13 and the receiver 14 are components of a reader configured to read the electromagnetic tag.

The reprocessing machine according to the present disclosure enable to implement a method for identifying and reprocessing a medical device ED, comprising:
/A/ opening the access door of a reprocessing machine 10
/B/ as the medical device ED is passing through the entry-opening EO and towards an inside of the washing chamber W, energizing the electromagnetic tag 1 with the energizing power source 13 of the reprocessing machine located at the access-door, and outside of the washing chamber W;
/C/ as the medical device ED is passing through the entry-opening EO and towards the inside of the washing chamber W and in response to the energizing, receiving an identification electromagnetic signal transferred by the energized electromagnetic tag 1 with the receiver 14 of the reprocessing machine located at the access-door 12 and outside of the washing chamber W.

By “at the access door”, the present disclosure means that the energizing power source 13 and the receiver 14 are positioned so as to read the electromagnetic tag 1, as the tag of the medical device ED crosses the level of the entry opening EO.

As shown for example on FIG. 1, the energizing power source 13 and the receiver 14 are positioned so as to read the electromagnetic tag 1 as the medical device is displaced towards the inside of the washing chamber along direction X. This position of reading is also shown on FIG. 3.

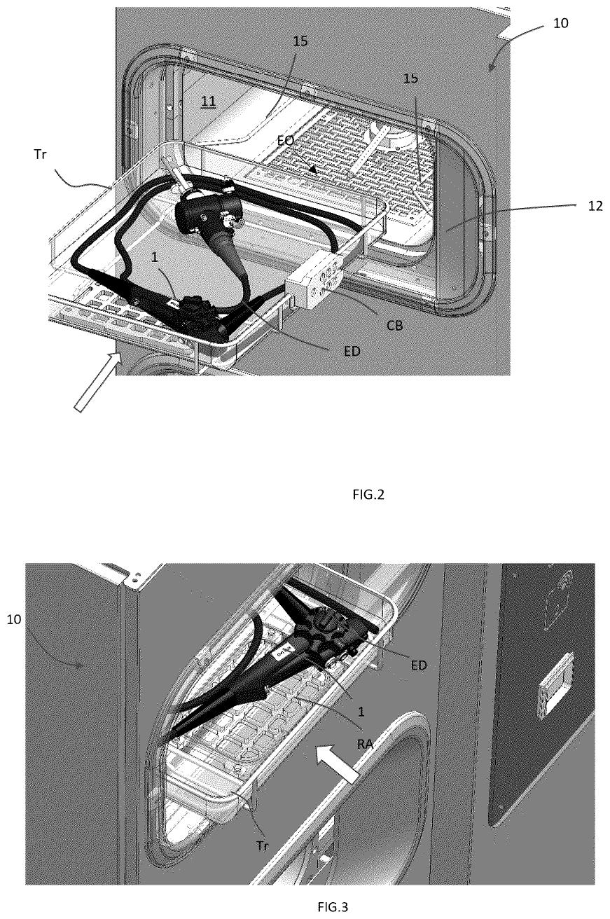

The energizing power source 13, and receiver 14 belonging to the reader may be positioned, in a fixed position relative to a door frame of the access door 12, possibly fixed to a lower beam of the door frame, as shown on FIG. 2.

As an antenna of the electromagnetic power source 13, and receiver 14 of the reader may typically be polarized, the antenna of the reader is typically orientated so that the maximum detection area of the reader lies in a zone at the level of the entry opening EO. For example, when the reader is fixed to the lower frame, the antenna is orientated so that the maximum detection lies vertically, according to direction Z, above the lower frame.

The position of the energizing power source 13 and the receiver 14 according to the present discosure enables the reader to read a tag, without the electromagnetic signal between the electromagnetic tag and the reader having to cross a wall of the tank. Thus, the tank can be fully in metal, in the meaning that the tank does not require having a wall part in plastic, permeable to the electromagnetic signal, to enable reading. Being able to have a tank fully in metal is advantageous compared to first prior art mentioned that requires a wall in plastic for reading the tag.

The technology used between the electromagnetic tag and the reader (comprising the energizing power source and the receiver) is a short-range technology. Short range technology only enables to read a tag efficiently and reliably when the electromagnetic tag is close to the reader; typically le reader and the tag being at a distance from each other below 15 cm, typically less than 10 cm.

For instance, for RFID tag, the orientation (pitch, yaw, and roll) of the tag relative to the antenna of the reader can affect the capacity of the reader to detect and read electromagnetic the tag. The technology used should, at least according to an example, being able to detect the tag, in different orientations of the tag (pitch, yaw, and roll) relative to the antenna of the reader 13, 14. The operator does not have to focus on having a specific orientation of the tag when crossing the area of detection of the reader so that the tag can be read reliably.

Advantageously, short-range technology forbids, or at least strongly limits the reader to detect and read an electromagnetic tag of a medical device which may be displaced in a same room where the reprocessing machine is located, but is not inserted through the entry-opening EB of the medical device. This is advantageous as it limits traceability error as compared to the second prior art mentioned that reads a tag of the medical device, when the access door is closed, well before the medical device is inserted in the trough an entry opening to the tank.

For instance, the electromagnetic tag may be a RFID tag, typically functioning at 13.56 MHZ (standard) for short range application. The frequency of the RFID tag may deviate from 13.56 MHZ as soon as it enables short range functioning, in the meaning explained above.

The reprocessing machine can typically comprises a control device including a processor and a storage and an instruction set configured to cooperate with the processor and the storage to:
/a/ open the access door 12 of a reprocessing machine;
/b/ energize the identification electromagnetic tag 1 with the energizing power source 13, as the medical device ED passes through the entry-opening and towards the inside of the washing chamber and,
/c/ receive an identification electromagnetic signal transferred by the energized electromagnetic tag with the receiver, as the medical device passes through the entry-opening and towards the inside of the washing chamber, and in response to the energization of the identification electromagnetic tag.

Instructions according to /a/ to open the access door 12, can be instructions controlling a motorized actuator configured to open the door, for instance a motorized sliding door. For examples, instructions according to /a/ can be triggered by the operator (or user) by a user interface. Motorized opening of the door from its closed position up to its open position is advantageous. For instance, it does not require the operator to touch the door. If the access door is opened manually, instructions according to /a/ can typically be instructions to unlock a motorized lock of the door. Once the motorized lock is opened, the reprocessing machine can typically display a message or a pictogram informing the operator that he may open the door manually.

The user interface to trigger opening of the door (or the lock) can advantageously be a contactless interface. For instance, the operator can have a personal tag (such as RFID, NFC, barre code, QR code) that can be read by a second reader of the reprocessing machine. The motorized opening of the door (or the motorized opening of the lock) may be triggered by an optic sensor pointing downward, as a foot of the operator cross a detection beam of the optic sensor. Such triggering techniques to initiate the opening of the access door are advantageous as they do not require the operator to touch the reprocessing machine. However, it is also possible to trigger the opening by having the operator contacting a pedal with his foot or having the operator pressing a button or having the operator touching a screen display.

Instructions according to /b/ to energize the power source and instructions according to /c/ to receive an identification are implemented after opening of the access door. The reader can be typically in a sleeping mode when the access door is closed, for instance to lower electrical energy consumption of the reprocessing machine.

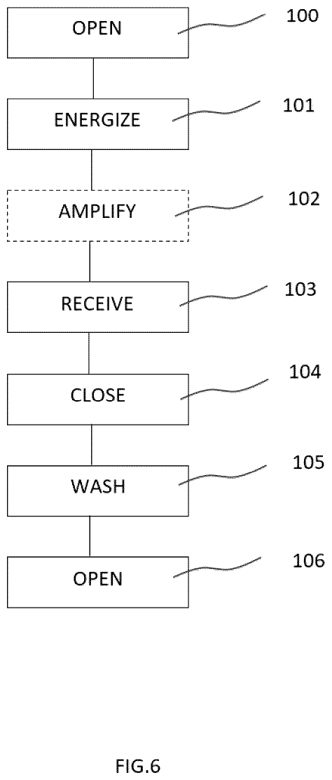

Diagram of FIG. 6 shows:
Opening according to /A/ on block 100,
Energizing according to /B/ on block 101,
Receiving according to /C/ on block 103.

As an example, the reprocessing machine 10 can further comprising a carrier device Tr for the medical device ED and supporting tracks 15 inside the tank for passing the medical device through the entry-opening OE towards the inside of the washing chamber.

The carrier device is typically a basket, or a perforated element that have voids (or holes) enabling to clean the underneath outer surface of the medical device resting on the carrier device, by spraying from below through the voids (of holes) of supporting surface of the carrier device.

As shown in example of FIG. 1, or in example of FIG. 2, 3, the tank has supporting track 15 oriented to enable to slide the carrier device Tr trough the entry opening EO. The sliding is oriented along direction X on FIG. 1. The supporting tracks 15 are typically positioned at a distance from the reader (comprising the energizing power source 13, and the receiver 14), in the vertical direction Z, to enable reading of the electromagnetic tag according to /B/ and/C/ of the method, as the medical device is resting on a supporting surface of the sliding carrier device Tr.

The method according to present disclosure can further comprise supporting the medical device with a carrier device Tr and sliding the carrier device along supporting tracks 15 inside the tank 11 for passing the medical device through the entry opening EO.

Such a carrier device Tr, and supporting track 15 enable the operator to insert the medical device ED through the entry opening EO, by grabbing manually the carrier device Tr, and thus to have the electromagnetic tag 1 read by the reader 13, 14, without having to grab manually the medical device ED. This is advantageous compared to first prior art that requires the operator to grab the medical device and to displace it against a reading zone of the wall of the tank, lateral to washing chamber. In such example of the present disclosure the operator does not have to touch the medical device for inserting the medical device in the washing chamber W.

For instance, the medical device ED is an endoscope having a plurality of lumens. The carrier device can have a coupling block CB enabling to fluidly connect the inner lumen with at least a part of the fluidic circuit that enables to clean the inner surfaces of the endoscope.

The coupling block CB has a plurality of through-holes that have an inlet side that are fluidly coupled to corresponding ones of the plurality of lumens and an outlet side configured to couple to a plurality of holes of the reprocessing machine.

The method according to the present disclosure can further comprise, before the opening the access door according to /A/;
coupling the plurality of lumens on the coupling block CB of the carrier device, the coupling block CB having a plurality of through-holes that have an inlet side that are fluidly coupled to corresponding ones of the plurality of lumens and an outlet side configured to couple to a plurality of holes of the reprocessing machine.

Once the carrier device is inserted in the washing chamber W, the method can comprise connecting the through holes of the coupling block CB with the holes of the reprocessing machine. Holes of the reprocessing machine are in communication with the part of the fluidic circuit that comprises valve(s) and pump(s) to enables the circulation of the cleaning solution through the inner lumens of the endoscope. The instruction set can further comprise instructions to couple the holes of the reprocessing machine with the outlet of the through hole, for instance by a motorized coupling.

The sprayer device can comprise a lower spraying device to the underneath surfaces of the medical device ED.

Lower spraying device can comprise a rotating sprayer arm 17. The rotating sprayer arm is located below the carrier device Tr supported by the supporting tracks 15. The rotating sprayer arm typically rotates according to a vertical axis of rotation.

The rotating sprayer arm 17 can be located above a level of the energizing power source 13 and the receiver 14, according to the vertical direction Z.

The sprayer device can also comprise an upper spraying device, fixed in the washing chamber on a roof of the tank. Upper spraying device sprays downwardly to clean the upper outer surfaces of the medical apparatus. Upper spraying device and lower spraying device enable to mechanically spray all the outer surfaces of the medical apparatus.

According to an example, and for instance due to the lower spraying device, and in particularly the rotating sprayer arm, the energizing power source 13 (and typically the receiver 14, and more generally the reader) is located at a vertical distance L from a supporting surface of the carrier device Tr supported by the tracks. The vertical distance can typically be greater than 4 cm and below 10 cm, for instance around 7 cm.

In particularly due to this distance, the carrier device can further comprise a relay antenna RA. Relay antenna is typically a passive antenna amplifier being located in between the identification electromagnetic tag 1 and the energizing power source 14, as shown on FIG. 1.

The relay antenna is configured to amplify the electromagnetic signal between the power source 13 and the electromagnetic tag 1, but also between the electromagnetic tag 1 and the receiver 14, as the medical device is passing through the entry-opening and towards the inside of the washing chamber.

The method according to the present disclosure can further comprise amplifying a signal of the energizing power source with the relay antenna RA of the carrier device Tr, to generate an amplified energizing signal reaching the electromagnetic tag as the medical device is passing through the entry-opening and towards the inside of the washing chamber, and in particularly during energizing according to /B/.

The method according to the present disclosure can further comprise amplifying the identification signal of the energized electromagnetic tag with the relay antenna RA of the carrier device Tr, to generate an amplified identification signal reaching the receiver 14 as the medical device is passing through the entry-opening and towards the inside of the washing chamber, and in particularly during receiving according to /C/.

Antenna of relay tag may be embedding in plastic, for example over molded by plastic. Plastic covering of antenna protect the antenna from cleaning liquid aggression. Relay antenna RA and its plastic embedding can form a grid (with holes or void), that enable the cleaning liquid spread from below to clean the underneath outer surfaces of the medical device.

Amplifying, as optional, is illustrated in dotted line in block 102 of FIG. 6.

A supporting wall of the carriers can thus comprise the plastic grid containing the antenna, in extension to a metallic support in a horizontal direction (along direction X and Y). Four rims can protrude vertically on four sides of the support wall of the carrier device (along direction Z), in order to maintain the medical device on the supporting surface of the supporting wall.

The method for identifying and reprocessing a medical device ED can further comprise:
/D/ closing the access door 12 in response passing the medical device through the entry opening and towards the inside of the washing chamber;
/E/ in response to closing the door, washing the medical device by applying a washing cycle by controlling the fluidic circuit comprising one or several pumps, one or several valves and a sprayer device located inside said washing chamber W.

Diagram of FIG. 6 shows:
closing according to /D/ on block 104,
washing according to /E/ on block 105.

Washing the medical device according to /E/ can comprise, at least in a first phase, spraying a cleaning solution, with the upper spraying device and the lower spraying device that enable to clean mechanically the outer surfaces of the medical device, with a least a first part of the fluidic circuit.

Washing the medical device according to /E/ can also comprise, at least in the first phase, circulating a cleaning solution into the plurality of lumens of the endoscope, in particular with the coupling block BC that is fluidly connected to the lumens, at the inlet of the plurality of through holes, and fluidly connected to the holes of the reprocessing machine, at the outlet of the plurality of through holes. Holes of the reprocessing machine are communicating with a second part of the fluidic circuit, that is typically independent to the first part of the circuit.

Closing the access-door 12, in a similar way to the opening can be manually operated, or be a motorized action.

A second phase of the washing cycle can comprise a drying step where air is blown on the outer surface and on the inner surface of the medical device to dry the surface, in particularly through the fluidic circuit.

The machine-readable storage medium can thus further comprise instructions to;
close the access door,
wash the medical device by applying a washing cycle by controlling a fluidic circuit comprising one or several pumps, on or several valves and a sprayer device located inside said washing chamber.

In general, the method for identifying and reprocessing a medical device ED can comprise, in response to receiving an identification electromagnetic signal, reading the identification information of the medical device, and comparing the identification information to stored information to determine if the medical device is authorized to be cleaned.

Thus, the machine-readable storage medium can further comprise instructions to;
read the identification information of the medical device, in response to receiving an identification electromagnetic signal, and,
compare the identification information to stored information to determine if the medical device is authorized to be cleaned.

If the medical device is not an authorized one, in response to the comparison, the washing according to /E/ is prevented, and possibly the closing of the access-door is also prevented. The method can comprise displaying on a screen of a user interface the information that the medical device should be removed from the tank.

In general, the method for identifying and reprocessing a medical device ED can advantageously comprise, in response to receiving an identification electromagnetic signal, reading the identification information of the medical device, and comparing the identification information to stored information to select wash parameters associated with the specific medical device inserted into the washing chamber.

Thus, the machine-readable storage medium can further comprise instructions to;
read the identification information of the medical device, in response to receiving an identification electromagnetic signal and,
compare the identification information to stored information to select wash parameters associated with the specific medical device inserted into the washing chamber.

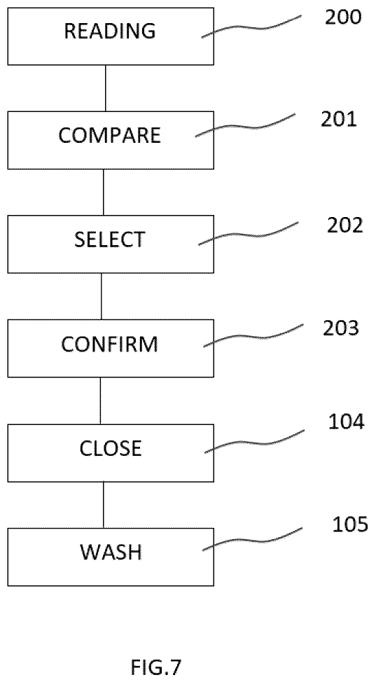

Diagram of FIG. 7 shows:
reading on block 200,
comparing on block 201.

Wash parameters can, for example only, comprises all or part of the following:
duration of washing cycle,
duration of spraying/circulating into the lumens the cleaning solution,
duration of drying
number of lumens,
pressure/flow rate of cleaning liquid. . . .

The machine-readable storage medium can thus comprise instructions to;
read the identification information of the medical device, in response to receiving an identification electromagnetic signal and,
compare the identification information to stored information to select wash parameters associated with the specific medical device inserted into the washing chamber.

According to an example, the selected wash cycle comprising the selected parameters can be initiated without having a confirmation of the user/operator.

According to another example, the selected wash cycle is initiated only after user/operator confirmation. In this case the method can further comprise:
displaying a selected wash cycle associated with the selected wash parameters on a screen of a user interface of the reprocessing machine, and waiting for a user confirmation to initiate the selected wash cycle;
initiating the selected wash cycle, in response to a user confirmation, typically on the user interface.

Confirmation of user is shown on block 203 of FIG. 7.

The user interface for user confirmation can be a contactless interface, such as having a personal user electromagnetic tag (such as RFID tag) read by a second reader.

The machine-readable storage medium can further comprise instructions to;
display a selected wash cycle associated with the selected wash parameters on a screen of a user interface of the reprocessing machine, and waiting for a user confirmation to initiate the selected wash cycle;
initiate the selected wash cycle, in response to a user confirmation, typically on the user interface.

In general, the closing of the access door according to /D/ can be triggered by user confirmation.

If the user discards confirmation, the method can enable the user to deviate from the selected wash cycle. The method can further comprise:
in response to a user discarding confirmation of the selected wash cycle, displaying on the screen of the user interface changes in wash parameters of the wash cycle, and allowing the user to select one or several changes in the parameters of the wash cycle,
initiating the wash cycle, in response to change(s) in the parameter, after a user confirmation on the user interface.

The machine-readable storage medium can further comprise instructions to;
in response to a user discarding confirmation of the selected wash cycle, display on the screen of the user interface changes in wash parameters of the wash cycle, and allowing the user to select one or several changes in the parameters of the wash cycle,
initiating the wash cycle, in response to change(s) in the wash parameters, after a user confirmation on the user interface.

In general, the machine-readable storage medium can further comprise instructions to update a traceability numerical file by changing a status of a corresponding identified medical device to a cleaned medical device.

Once the washing cycle is finished the method can further comprise:
/F/ extracting the washed medical device through an exit door. According to a non-shown example exit door can be the same door as the access-door 12.

According to another example, for instance the exit door 16 is distinct to access-door 12, located opposite to the access door relative to the washing chamber W. Once opened exit door frees an exit opening authorizing the medical device to be extracted.

The reprocessing machine can be a pass-through type of machine, the entry opening communicating with a first room, typically a dirty room, the exit opening, opposite, communicating with a second room, typically a clean room (for which a controlled atmosphere is typically purified by filtration and in (slight) over-pressure relative to the dirty room), the reprocessing machine lying in aperture in a wall separating the two rooms.

As to the closing or opening of the access door, the closing and opening of the exit door can be manually operated, in particularly once a motorized lock has been opened. Advantageously, the closing and opening of the exit door 16 may be motorized to pass from the opened position to the closed position, or inversely from the closed position up to the opened position.

In general, the method and reprocessing machine have been described and illustrated, as washing a single medical device in the washing chamber of the reprocessing machine.

The method and reprocessing machine are not limited to wash a single medical device, as the present disclosure may enable to wash simultaneously several medical devices (for instance two endoscopes) into the washing chamber, in particular resting both on the carrier device.

As a best mode, the method for identifying and reprocessing a medical apparatus, and the reprocessing machine as such, can enable the operator (or user) to implement all blocks (100 to 106) of diagram of FIG. 6, without the operator having to touch the medical device, or the reprocessing machine, in particularly if the operator accepts by a contactless interface the selected washing cycle.

### REFERENCE SIGNS LIST

1. Electromagnetic tag,
10. Reprocessing machine,
11. Tank,
12. Access-door,
13. Energizing power source (for energizing the tag),
14. Receiver (for receiving identification signal transferred by the tag),
15. Supporting tracks,
16. Exit door,
17. Rotating spraying arm,
ED. Medical device,
EO. Entry opening,
W. Washing chamber
Tr. Carrier device (for supporting the medical device),
CB. Coupling block,
RA. Relay antenna

1. A method for identifying and reprocessing a medical device, the medical device being equipped with an electromagnetic tag, the electromagnetic tag comprising an antenna and a memory comprising information on the medical device, said method comprising:
opening an access door of a reprocessing machine comprising a tank delimiting a washing chamber, the access door being configured to close an entry-opening of the tank for passing the medical device through the entry-opening;
as the medical device is passing through the entry-opening and towards an inside of the washing chamber, energizing the electromagnetic tag with an energizing power source of the reprocessing machine located at the access door, and outside of the washing chamber; and
as the medical device is passing through the entry-opening and towards the inside of the washing chamber and in response to the energizing, receiving an identification electromagnetic signal transferred by the energized electromagnetic tag with a receiver of the reprocessing machine located at the access-door and outside of the washing chamber.

2. The method of claim 1, further comprising, in response to receiving an identification electromagnetic signal, reading the identification information of the medical device, and comparing the identification information to stored information to determine if the medical device is authorized to be cleaned.

3. The method of claim 1, further comprising, in response to receiving an identification electromagnetic signal, reading the identification information of the medical device, and comparing the identification information to stored information to select wash parameters associated with the specific medical device inserted into the washing chamber.

4. The method of claim 3, further comprising:
displaying a selected wash cycle associated with the selected wash parameters on a screen of a user interface of the reprocessing machine and waiting for a user's confirmation to initiate the selected wash cycle; and
initiating the selected wash cycle, in response to a user confirmation on the user interface.

5. The method of claim 4, further comprising:
in response to a user discarding confirmation of the selected wash cycle, displaying on the screen of the user interface changes in wash parameters of the wash cycle, and allowing the user to select one or several changes in the parameters of the wash cycle; and
initiating the wash cycle, in response to change in the parameter, after a user confirmation on the user interface.

6. The method of claim 1, further comprising supporting the medical device with a carrier device and sliding the carrier device along supporting tracks inside the tank for passing the medical device through the entry opening.

7. The method of claim 1, wherein the medical device is an endoscope having a plurality of lumens, and wherein the method further comprises, before the opening the access door:
coupling the plurality of lumens on a coupling block of the carrier device, the coupling block having a plurality of through-holes that have an inlet side that are fluidly coupled to corresponding ones of the plurality of lumens and an outlet side configured to couple to a plurality of holes of the reprocessing machine.

8. The method of claim 1, further comprising amplifying a signal of the energizing power source with a relay antenna of the carrier device, the relay antenna being located between the identification electromagnetic tag and the energizing power source, as the medical device is passing through the entry-opening and towards the inside of the washing chamber, so as to generate an amplified energizing signal reaching the electromagnetic tag.

9. The method according of claim 1, wherein the electromagnetic tag is a RFID Tag.

10. The method of claim 1, further comprising:
closing the access door in response passing the medical device through the entry opening and towards the inside of the washing chamber; and
in response to closing the door, washing the medical device by applying a washing cycle by controlling a fluidic circuit comprising one or several pumps, one or several valves and a sprayer device located inside said washing chamber.

11. The method of claim 1, further comprising extracting a washed medical device through an exit door located opposite to the access door relative to the washing chamber.

12. A reprocessing machine configured to clean a medical device, the medical device being equipped with an electromagnetic tag, the reprocessing machine comprising:
a tank delimiting a washing chamber, and an access door configured to close an entry-opening of the tank for the medical apparatus;
a fluidic circuit comprising one or several pumps, one or several valves, and a sprayer device inside the washing chamber;
an energizing power source configured to energize the identification electromagnetic tag, and a receiver configured to receive an identification electromagnetic signal of the electromagnetic tag, in response to the energization, the energizing power source and the receiver being located at the access-door outside the washing chamber; and
a control device including a processor, a storage, and an instruction set configured to cooperate with the processor and the storage to:
open the access door of a reprocessing machine;
energize the identification electromagnetic tag with the energizing power source, as the medical device passes through the entry-opening and towards the inside of the washing chamber; and
receive an identification electromagnetic signal transferred by the energized electromagnetic tag with the receiver, as the medical device passes through the entry-opening and towards the inside of the washing chamber, and in response to the energization of the identification electromagnetic tag.

13. The reprocessing machine of claim 12, further comprising a carrier device for the medical device and supporting tracks inside the tank for passing the medical device through the entry-opening towards the inside of the washing chamber.

14. The reprocessing machine of claim 13, further comprising a relay antenna of the carrier device, the relay antenna being located in between the identification electromagnetic tag and the energizing power source, as the medical device is passing through the entry-opening and towards the inside of the washing chamber, so as to generate an amplified energizing signal reaching the electromagnetic tag.

15. The reprocessing machine of claim 13, wherein the medical device is an endoscope having a plurality of lumens, and wherein the carrier device has a coupling block, the coupling block having a plurality of through-holes that have an inlet side that are fluidly coupled to corresponding ones of the plurality of lumens and an outlet side configured to couple to a plurality of holes of the reprocessing machine.

16. The reprocessing machine of claim 12, wherein the sprayer device comprises a rotating sprayer arm, the rotating sprayer arm being located:
below the carrier device supported by the supporting tracks; and
above a level of the energizing power source and the receiver.

17. The reprocessing machine of claim 12, wherein the energizing power source is located at a vertical distance from a supporting surface of the carrier device supported by the tracks, the vertical distance being greater than 4 cm and below 10 cm.

18. The reprocessing machine of claim 12, wherein the energizing power source and the receiver are fixed to a lower beam of a door frame of the access-door.

19. A non-transitory machine-readable storage medium encoded with instructions executable by a processor, the machine-readable storage medium comprising:
instructions to open an access door of a reprocessing machine;
instructions to energize the electromagnetic tag with the energizing power source, as the medical device passes through the entry-opening and towards the inside of the washing chamber; and
instructions to receive an identification electromagnetic signal transferred by the energized electromagnetic tag with the receiver, as the medical device s passes through the entry-opening and inside the washing chamber, and in response to the energization of the identification electromagnetic tag.

20. The non-transitory machine-readable storage medium of claim 19, the machine-readable storage medium further comprising instructions to:
close the access door; and
wash the medical device by applying a washing cycle by controlling a fluidic circuit comprising one or several pumps, on or several valves and a sprayer device located inside said washing chamber.

21. The non-transitory machine-readable storage medium of claim 19, the machine-readable storage medium further comprising instructions to:
read the identification information of the medical device, in response to receiving an identification electromagnetic signal; and
compare the identification information to stored information to determine if the medical device is authorized to be cleaned.

22. The non-transitory machine-readable storage medium of claim 19, the machine-readable storage medium further comprising instructions to:
read the identification information of the medical device, in response to receiving an identification electromagnetic signal; and
compare the identification information to stored information to select wash parameters associated with the specific medical device inserted into the washing chamber.
